(19) United States
(12) Patent Application Publication
(10) Pub. No.: US 2013/0131380 A1
(43) Pub. Date: May 23, 2013

(54) PROCESS FOR THE SELECTIVE OXIDATION OF CARBON MONOXIDE

(71) Applicants: Jean-Luc Dubois, Millery (FR); Nicolas Dupont, Saint Avold (FR); Gregory Patience, Quebec (CA)

(72) Inventors: Jean-Luc Dubois, Millery (FR); Nicolas Dupont, Saint Avold (FR); Gregory Patience, Quebec (CA)

(73) Assignee: Arkema France, Colombes (FR)

(21) Appl. No.: 13/638,682

(22) PCT Filed: Mar. 29, 2011
(86) PCT No.: PCT/FR2011/050692

§ 371 Date: Oct. 18, 2012

(30) Foreign Application Priority Data
Mar. 30, 2010 (FR) 1052306

(51) Int. Cl. C07C 51/15

(52) U.S. Cl. CPC C07C 51/15
USPC 562/545; 423/437.2

(57) ABSTRACT

The invention relates to a process of the selective oxidation of carbon monoxide to carbon dioxide present in a gas mixture comprising at least one hydrocarbon or a hydrocarbon derivative, and to its integration into a process for producing hydrocarbon derivatives. The process according to the invention comprises a step that consists in bringing said gas mixture into contact with a solid catalyst capable of oxidizing carbon monoxide to carbon dioxide at a chosen temperature, characterized on that said step is carried out in a fluidized bed.

### FIELD OF THE INVENTION

The invention relates generally to the field of the production of hydrocarbon derivatives from hydrocarbons in the gas phase in the presence of oxygen or of an oxygen-comprising gas. More specifically, the invention relates to a process for the selective oxidation of carbon monoxide to give carbon dioxide present in a gas mixture comprising at least one hydrocarbon or one hydrocarbon derivative, and to its incorporation in a process for the production of hydrocarbon derivatives.

### PRIOR ART AND TECHNICAL PROBLEM

Numerous hydrocarbon derivatives are produced industrially by partial oxidation of an appropriate hydrocarbon in the gas phase in the presence of molecular oxygen or of a gas comprising molecular oxygen and of a suitable catalyst.

For example, the main process for the production of acrylic acid is based on the oxidation of propylene and/or propane. The synthesis of acrylic acid by oxidation of propylene comprises two stages; the first is targeted at the oxidation of the propylene to give acrolein and the second at the oxidation of the acrolein to give acrylic acid. This synthesis is generally carried out in two reactors using two catalytic systems specific for each of the oxidation stages, the two stages being carried out in the presence of oxygen or of an oxygen-comprising gas.

In the same way, methacrolein and methacrylic acid are produced industrially by catalytic oxidation of isobutene and/or tert-butanol.

Anhydrides, such as maleic anhydride or phthalic anhydride, can be produced, by catalytic oxidation of aromatic hydrocarbons, such as benzene or o-xylene, or of straight-chain hydrocarbons, such as n-butane or butene.

Acrylonitrile is produced by catalytic oxidation in the gas phase of propylene or propane by air in the presence of ammonia, such a reaction being known under the name of ammoxidation. Similarly, the ammoxidation of isobutane/isobutene or methylstyrene results respectively in methacrylonitrile and atroponitrile.

These various processes are carried out in the gas phase, in the presence of molecular oxygen or of a gas comprising molecular oxygen, generally in the presence of air for economic reasons. Secondary reactions also take place during the catalytic oxidation reactions, including the reaction for the final oxidation of the reactant, resulting in the formation of carbon oxides and water.

Thus, these various processes for the manufacture of hydrocarbon derivatives by selective oxidation have in common the fact that they generate the formation of carbon oxides, carbon monoxide CO and carbon dioxide CO₂. Carbon monoxide CO is generally produced in excess with respect to the CO₂. They are gaseous compounds which are noncondensable under the temperature and pressure conditions normally employed in the stages for the recovery/purification of the hydrocarbon derivatives. For this reason, they are discharged to the atmosphere in the form of vents for the incineration of the streams removed during the process, with the disadvantage of emitting large amounts of CO₂ to the atmosphere.

As in the majority of industrial processes, it is advantageous to recycle some gas fractions generated during the manufacture of hydrocarbon derivatives, in particular the gas fraction comprising the unreacted hydrocarbon, for obvious reasons of productivity and profitability. In the case of the presence of carbon monoxide in these recycled gases, there is a risk of this noncondensable compound accumulating during the process and causing safety problems, in particular risks of loss of control of the oxidation reaction and of explosion in the oxidation reactor, carbon monoxide being flammable. Furthermore, carbon monoxide may have a harmful effect on the oxidation catalysts; in particular, it is a poison for many catalysts, including the conventional catalysts for the oxidation of propylene to give acrolein.

One solution for overcoming these disadvantages was provided in the document EP 484 136, which describes a process for the production of a hydrocarbon derivative comprising the recycling of the gas stream resulting from the reaction, after having successively extracted or separated, from this stream, the expected hydrocarbon derivative, converted the CO present in this gas stream into CO₂ and then withdrawn a portion of the CO₂ formed. The recycled stream is thus a stream depleted in CO and CO₂. The reaction for the oxidation of the hydrocarbon is carried out in the presence of an inert diluent, in particular in the presence of CO₂, at contents sufficiently high to prevent the formation of a flammable mixture in the system. The catalyst, for the selective oxidation of the CO to CO₂ is chosen from catalysts which are nonoxidizing with respect to the unreacted hydrocarbon present in the gas stream, generally catalysts based on copper/manganese or platinum/nickel mixed oxides, optionally supported on silica or alumina. The examples illustrating this process are carried out with a CO converter comprising a fixed catalyst bed. In the case of a process for the production of acrylic acid from propylene, the tests indicate a degree of 80% for the conversion of the CO but also a conversion of a portion or the propylene and propane, thus generating losses of reactant in the process.

Another solution for overcoming these disadvantages was provided in the document EP 1 007 499 relating to a high-yield process for the production of maleic anhydride from n-butane, consisting in bleeding off a fraction of the recycling gases, so as to prevent inert gases and carbon oxides from accumulating, and in selectively oxidizing the carbon monoxide present in the bleed stream to carbon dioxide in the presence of a catalyst capable of selectively oxidizing the CO to CO₂ in a gas having an O₂/CO molar ratio of between 0.5 and 3. Catalysts which can be used in this case for the selective oxidation of the CO to CO₂ consist, for example, of supported precious metals, such as platinum, rhodium, ruthenium or palladium. The CO converter is preferably of fixed bed tubular type.

The document EP 1 434 833 provides, as catalyst for the selective oxidation of CO to CO₂ in a gas stream comprising at least one alkane, a catalyst substrate, such as Pt, Pd, Pt—Fe or Pd—Fe, on a silica support, provided, with a virtually continuous coating of a material consisting of a molecular sieve. The selective oxidation of the CO is carried out in a fixed bed at a temperature chosen so that the catalyst does not have any effect on the alkane.

In the process for the production of methacrylic acid from isobutane described in the document EP 495 504, use is made, after separation of the condensable fraction comprising the methacrylic acid, of a stage of oxidation of the CO present in the noncondensable gas stream in order to convert it to CO₂, followed by a stage of separation of the CO₂ in the carbonic anhydride form by absorption in a liquid comprising, for example, a solution of potassium carbonate and an amine. Catalysts which can be used for the conversion of the CO to CO₂, without oxidation of the isobutane, for example comprise palladium and/or platinum on a support, gold on a support, or manganese oxide. In order to remove the heat of the oxidation reaction, a reactor of multitubular type is used for the CO converter.

In these various processes of the state of the art, the selective oxidation of the CO to CO₂ is carried out either, on the one hand, at low space velocities SVs (generally less than 10 000 h⁻¹), the space velocity being defined as the ratio of the flow rate of reactants to the volume of catalyst, or as an inverse of the “contact time”, or, on the other hand, for low concentrations of CO at the inlet; in some cases, it also results in a simultaneous conversion of the hydrocarbon present in the gas.

Due to the risks of flammability associated with the presence of hydrocarbons and oxygen, processes for the production of hydrocarbon derivatives by catalytic oxidation in the gas phase of hydrocarbons generally operate with low contents of hydrocarbons in order to keep the gaseous reaction mixture outside the flammability region.

In order to increase the productivity of these processes while observing safety constraints, it is possible to carry out the reaction of the oxidation of the hydrocarbon in the presence of an inert gas with a high specific heat (also known as specific heat capacity Cp, the index p indicating a value at constant pressure), in order to obtain better management of the exothermicity of the reaction and to increase the concentration of the hydrocarbon in the reaction mixture, the inert gas constituting a thermal ballast.

Thus, the document EP 293 224 provided for the addition of 5 to 70% by volume of a saturated aliphatic hydrocarbon of 1 to 5 carbon atoms, such as methane, ethane or propane, for the reaction of the oxidation of propylene resulting in acrolein in a two-stage process for the production of acrylic acid by catalytic oxidation, the second stage consisting in oxidizing the acrolein to acrylic acid.

Propane is preferred as hydrocarbon which can be used as thermal ballast in this process. This is because this gas ballast exhibits several advantages with respect to a ballast formed of inert gases, such as nitrogen or carbon dioxide. First, it creates a better thermal ballast as its specific heat (Cp) increases strongly with the temperature, which is not the case with nitrogen. In addition, it has a degree of chemical inertia under the conditions under which the reaction for the oxidation of propylene is carried out and its possible reaction products are very similar in nature to those of the propylene. Finally, it makes it possible to more easily meet the constraints of composition of the mixture related to the question of the flammability by placing the reaction mixture above the upper flammability limit. By virtue of the use of this thermal ballast, the feedstock supplying the reactor for the oxidation of propylene can be greater as fraction by volume, which increases the productivity of the conversion while controlling the hot spots in the bed of catalysts and thus promoting the selectivity of the reaction.

It is even more necessary to recycle the stream comprising the unreacted propylene (and the propane) in the case of the use of a thermal ballast such as propane, due to the cost of the propane, and consequently the oxidation of the carbon monoxide in a stream rich in mixtures of hydrocarbons becomes more difficult to carry out selectively.

The problem which the present invention intends to solve is that of carrying out the conversion of the CO to CO₂ in a stream rich in hydrocarbons or in a mixture of hydrocarbons, without, however, oxidizing these hydrocarbons. It is possible, in an adiabatic fixed bed or even in a reactor of the multitubular type with good neat exchange, for selective conversion of the CO to start but the reaction can rapidly run away, which greatly increases the temperature of the catalyst and brings about the conversion of the other compounds present in the stream. Specifically, in a fixed bed reactor, the large amounts of CO present lead to an adiabatic increase in the temperature such that the temperatures for the start of conversion (ignition temperatures) of all the constituents are exceeded.

The exothermicity of the reaction for the conversion of CO to CO₂ is characterized by ΔHr of 283 kJ/mol. Even if the exothermicity of the reaction for the selective oxidation of hydrocarbon is comparable, for example ΔHr is 341 kJ/mol for the conversion of propylene to acrolein, the kinetics of the combustion reaction are much faster, A “conventional” technology of the multitubular reactor type then does not make it possible to correctly control the temperature of the catalyst. Under conditions where the temperature can be controlled, the conversion of the CO to CO₂ can be carried out at a lower temperature than the temperature for oxidation of the hydrocarbon and thus it can be carried out selectively.

As the differences in ignition temperature between CO and the various constituents present in a stream of hydrocarbons are significant, it is theoretically possible to selectively oxidize CO to CO₂ provided chat the reaction temperature is maintained between the ignition temperature of CO and that of the other constituents.

By way of indication, the increase in the temperature of the gas during the combustion of 1000 ppm of impurity in air is illustrated in the following table 1.

TABLE 1

| | | Adiabatic |
|---|---|---|
| | Heat of | temperature |
| | combustion | increase of the |
| Substance | kcal/mcl | gas for 1000 ppm |
| Benzene | 750.6 | 103 |
| Toluene | 892.0 | 123 |
| m-Xylene | 1038.9 | 143 |
| Methyl Ethyl Ketone | 540.1 | 74 |
| Methyl Isobutyl Ketone | 843.0 | 116 |
| Methanol | 149.8 | 21 |
| Formaldehyde | 124.0 | 17 |
| Acrolein | 369.0 | 51 |
| Acetic Acid | 188.3 | 26 |
| Butyric Acid | 482.0 | 66 |
| Ethyl Acetate | 494.7 | 68 |
| Phenol | 690.0 | 95 |
| m-Cresol | 838.0 | 115 |
| Acetaldehyde | 257.8 | 35 |
| Styrene | 1005.0 | 138 |
| Hydrogen sulfide | 122.5 | 17 |
| Ammonia | 76.2 | 11 |
| Trimethylamine | 531.0 | 73 |
| Carbon Monoxide | 67.6 | 9 |

In an atmosphere rich in gas with a higher specific heat than that of the air, such as propane, the increase in adiabatic temperature is proportionally lower.

In this context, the applicant company has discovered that the use of a fluidized bed to convert the CO to CO₂ in a stream rich in hydrocarbons makes it possible to solve the various problems mentioned above and to optimally meet the requirements of use of thermal ballast and/or of recycling of reaction gas comprising an excessively high CO content, in processes for the production of hydrocarbon derivatives by selective oxidation of hydrocarbons in the gas phase in the presence of oxygen and more generally in processes for the production of hydrocarbon derivatives from hydrocarbons in the gas phase in the presence of oxygen.

The oxidation of CO using a fluidized bed has already formed the subject of studies, in particular for studying the influence of the configuration of the reactor comprising a specific catalyst of Pt—Co—Ce/γ-Al₂O₃ type on the conversion of the CO present, in a hydrogen-rich stream (M. P. Lobera et al., Catalysis Today, 157 (2010), 404-409). In this study, the treated stream comprises only hydrogen, so that the problems of selective oxidation of CO in a complex mixture, such as a mixture of hydrocarbons, the oxidation of which has to be avoided, are not posed.

The aim of the present invention is thus to provide a process for the selective oxidation of carbon monoxide to carbon dioxide which is easily incorporated in existing industrial processes for the production of hydrocarbon derivatives.

### SUMMARY OF THE INVENTION

A subject matter of the present invention is thus a process for the selective oxidation of the carbon monoxide present in a gas mixture comprising at least one hydrocarbon or one hydrocarbon derivative, said process comprising the stage consisting in bringing said gas mixture into contact with a solid catalyst capable of oxidizing the carbon monoxide to carbon dioxide at a chosen temperature, characterised in that said stage is carried out in a fluidized bed.

The fluidized bed technology provides mixing of the solid and the gas mixture, and thus homogenization of the temperature of the catalyst. By obtaining a homogeneous temperature, it is thus possible to control the selectivity of the reaction for the oxidation of the carbon monoxide and to no longer destroy the molecules of economic value. This homogeneity confers, on the fluidized bed, an undeniable advantage in comparison with the fixed beds, which are generally subject to a high temperature gradient. The removal of the heat of the reaction can be provided by cooling pins positioned in the fluid bed. The coefficient for transfer of heat between the suspension and the exchanger tubes is very high, and makes it possible to efficiently heat or cool the material.

The process for the selective oxidation of CO according to the invention can be incorporated in any industrial process requiring bleeding of CO₂ and/or CO for chemical reasons (inhibition of the reaction), physical reasons (decrease in the Cp of the reaction gas) or safety reasons (flammability limits).

Another subject matter of the invention, is a process for the production of a hydrocarbon derivative comprising at least one stage of selective oxidation of the carbon monoxide present in a gas mixture comprising at least one hydrocarbon or one hydrocarbon derivative using a fluidized bed comprising a solid catalyst capable of oxidizing carbon monoxide to carbon dioxide at a chosen temperature.

Other characteristics and advantages of the invention will more clearly emerge on reading the detailed description which follows and the nonlimiting implementational examples of the invention.

### DETAILED DESCRIPTION

The gas mixture subjected to the process for the selective oxidation of CO according to the invention comprises at least one hydrocarbon or one hydrocarbon derivative.

The hydrocarbons are saturated or mono- or diunsaturated and linear or branched hydrocarbons comprising from 2 to 6 carbon atoms, or aromatic hydrocarbons, which can be substituted, comprising from 6 to 12 carbon atoms.

Mention may be made, as examples of hydrocarbons, for example, of ethylene, propane, propylene, n-butane, isobutane, isobutene, butene, butadiene, isopentene, benzene, o-xylene, methylstyrene or naphthalene. Preferably, the hydrocarbon is chosen from propylene or propane, alone or as a mixture.

The hydrocarbon derivative can be a product of the partial oxidation of a hydrocarbon and it can then be chosen from anhydrides, such as phthalic anhydride or maleic anhydride, aldehydes, such as acrolein or methacrolein, unsaturated carboxylic acids, such as acrylic acid or methacrylic acid, unsaturated nitriles, such as acrylonitrile, methacrylonitrile or atroponitrile, or their mixtures. Preferably, the hydrocarbon derivative is acrolein and/or acrylic acid.

The hydrocarbon derivative can also be a product of the addition of oxygen or a halogen compound to an unsaturated hydrocarbon, for example ethylene oxide, propylene oxide or 1,2-dichloroethane.

Mention may be made, as solid catalysts capable of oxidizing carbon monoxide to carbon dioxide which can be used in the process according to the invention, of known catalysts for the selective oxidation of CO, such as, for example, without this list being limiting, catalysts based on noble metals, such as platinum, palladium, rhodium or ruthenium, supported on an inorganic support, such as silica, titanium oxide, zirconium oxide, alumina or silicalite; or catalysts based on copper, manganese, cobalt, nickel or iron, optionally in the presence of at least one noble metal, such as platinum, palladium, rhodium or ruthenium, in the form of mixed oxides or of alloys optionally supported on an inorganic support, such as silica, titanium oxide, zirconium oxide, alumina or silicalite. Highly suitable catalysts are, for example, solids with a low charge of platinum or palladium (for example of the order of 2%; on a support of silicalite or sodium silicate type.

The catalyst employed in the process according to the invention is in the form of solid particles with a particle size ranging from 20 to 1000 microns, preferably from 40 to 500 microns, more particularly from 60 to 200 microns. The size distribution of the particles can be determined according to numerous methods, in particular according to a simple method, such as sieving with a sequence of sieves of decreasing mesh sizes, or determination by laser diffraction, for example with devices of the Malvern brand.

According to the invention, the temperature of the fluidized bed is between 20° C. and 400° C., preferably between 70° C. and 300° C., more preferably between 100° C. and 230° C.

Preferably, a temperature is chosen which is lower than the “ignition” temperature of the hydrocarbon and/or of the hydrocarbon derivative present in the gas mixture, that is to say lower than the temperature corresponding to the start of the reaction for the oxidation of the hydrocarbon and/or hydrocarbon derivative.

For example, in the case of propylene, there is generally at least approximately 20° C. and preferably at least 30° C. difference between the ignition temperature of CO (beginning of combustion of the CO) and the ignition temperature of propylene. This difference is sufficient to guarantee combustion of the CO without having combustion of the propylene. If the ignition temperature of propylene is reached, the reaction becomes difficult to control due to the high oxygen and propylene content of the gas to be treated.

The fluidized bed can operate batchwise or continuously (semibatchwise or open). Preferably, the process according to the invention is carried out continuously. This is because, given the ease of withdrawal of solid particles from the fluidized bed and of addition of solid particles to the fluidized bed during its operation, the solid phase can be continually replaced as required. The catalyst bed can maintain an unvarying activity over time if deactivated catalyst is continually withdrawn in order to replace it with fresh catalyst. The emptying and the cleaning of the fluidized beds take place very easily, as for a water tank. The withdrawal operations can be carried out continuously or with a degree of periodicity. Furthermore, the deactivated catalyst can optionally be reactivated ex situ by any appropriate technique, in order to be subsequently reinjected into the reactor. Mention may be made, as techniques for reactivation of the catalyst, without being limiting, of redispersion of the metals of the catalyst by a reducing treatment, washing the catalyst in order to remove the contaminants, or reimpregnation of the catalyst with a fresh charge of the active metals of the catalyst.

Reference may be made to the document on fluidization techniques in Techniques de l'Ingénieur [Techniques of the Engineer] J3 390 1 to 20.

Advantageously, mild oxidation conditions are used, that is to say a combination of a relatively inactive catalyst (comprising a low charge of active metal), a moderate temperature (for example from 80° C. to 180° C.) and a fairly short contact time (for example of less than one second). Alternatively, a low residence time makes it possible to envisage a higher temperature for a given catalyst.

Advantageously, use is made of a moderate pressure in the fluidized bed reactor, for example of between 1 and 3 bar, and a fairly short residence time, which is reflected by high space velocities. In the case where the conditions related to the process require higher pressures, it is then preferable to combine a relatively inactive catalyst with a temperature which is as low as possible in order to limit the reactions for the oxidation of the other constituents of the gas stream.

The linear velocity of the gas mixture in the fluidized bed can range from 0.1 to 80 cm/s. For fluidized beds of industrial size, it can range from 50 to 80 cm/s. In the case of shorter fluidized beds, in particular laboratory fluidized beds, the linear velocity of the gases is generally between 0.1 and 10 cm/s.

Generally, the flow rate by volume of the gas stream will be adjusted to the volume of catalyst and consequently to the size of the reactor, so as to achieve very high space velocities SVs, expressed as hourly flow rate by volume of reactants with respect to the volume of catalyst.

The process according to the invention is advantageously carried out with high space velocities SVs, for example ranging from 1000 h⁻¹ to 30 000 h⁻¹, preferably greater than 10 000 h⁻¹, or better still ranging from 10 000 h¹⁻¹ to 30 000 h⁻¹. The process according to the invention is particularly well suited to gas streams comprising more than 0.5 mol % of carbon monoxide at the inlet and preferably more than 1 mol % of carbon monoxide.

Another subject matter of the invention is a process for the production of a hydrocarbon derivative comprising at least the following stages:

a) at least one hydrocarbon, and oxygen or an oxygen-comprising gas are brought into contact with an appropriate catalyst, resulting in a gas mixture comprising at least one hydrocarbon derivative, unconverted hydrocarbon, oxygen and carbon monoxide,
b) the hydrocarbon derivative is separated or extracted from the reaction stream resulting from stage a),
c) the carbon monoxide present in the gas stream is then converted to carbon dioxide using a fluidized bed comprising a solid catalyst capable of oxidizing carbon monoxide to carbon dioxide at a chosen temperature, producing a gas stream depleted in carbon monoxide,
d) said stream depleted in carbon monoxide is recycled to reaction stage a).

It is clearly understood that this process can comprise other stages preliminary, intermediate and/or subsequent to those mentioned above which are well known to a person skilled in the art.

According to the process of the invention, stage a) is carried out under appropriate conditions, in particular regarding the nature of the catalyst, the temperature and the optional presence of an inert gas as thermal ballast, according to processes known to a person skilled in the art which make it possible to manufacture the desired hydrocarbon derivative.

The reaction carried out can be an oxidation reaction or a reaction for the addition of oxygen to an unsaturated hydrocarbon.

In an alternative form of the invention, stage a) is carried out in the presence of a thermal ballast which is inert under the conditions of the reaction carried out.

Stage b) consists in recovering the hydrocarbon derivative according to conventional methods using techniques such as absorption in a solvent followed by extraction, distillation, crystallization, condensation.

On conclusion of this stage b), the gas stream, freed from most of the hydrocarbon derivative, generally comprising unconverted hydrocarbon, oxygen, water vapor, inert gases, such as nitrogen and argon, carbon monoxide and carbon dioxide, is brought into contact with a solid catalyst capable of oxidizing the carbon monoxide to carbon dioxide at a chosen temperature, in a fluidized bed (stage c)), resulting in a stream depleted in carbon monoxide, which can be recycled, according to stage d), to the reaction stage a), optionally after having bled it of a portion of the carbon dioxide formed.

In a specific embodiment of the invention, the process for the production of a hydrocarbon derivative incorporating a stage c) of partial oxidation of CO to CO₂ using a fluidized bed relates to the manufacture of acrylic acid by catalytic oxidation of propylene using oxygen or an oxygen-comprising mixture and in the presence of propane as thermal ballast.

This reaction, widely operated industrially, is generally carried out in the gas phase and generally in two stages:

The first stage carries out the substantially quantitative oxidation of the propylene to give an acrolein-rich mixture, in which the acrylic acid is a minor component, and then during the second stage carries out the selective oxidation of the acrolein to give acrylic acid.

The reaction conditions of these two stages, carried out in two reactors in series or in a single reactor comprising the 2 reaction stages in series, are different and require catalysts appropriate to the reaction; however, it is not necessary to isolate the first-stage acrolein during this two-stage process.

The reaction conditions of these two stages, carried out in two reactors in series or in a single reactor comprising the 2 reaction stages in series, are different and require catalysts appropriate to the reaction; however, it is not necessary to isolate the first-stage acrolein during this two-stage process.

The reactor can be supplied with a propylene feedstock of low purity, that is to say comprising propane, such that the propane/propylene ratio by volume is at least equal to 1. As the large gas ballast formed by the propane results in a better management of the exothermicity of the reaction, the reactor can be supplied with a feedstock more concentrated in propylene in order to increase the productivity of the process. The other components of the reactive stream can be inert compounds, such as nitrogen or argon, water and oxygen.

The gas mixture resulting from the reaction for the oxidation of acrolein consists, apart from acrylic acid:
of light compounds which are noncondensable under the temperature and pressure conditions normally employed: nitrogen, unconverted oxygen, unconverted propylene, propane present in the propylene or added, as thermal ballast, carbon monoxide and carbon dioxide, which are formed in a small amount by final oxidation or going around in circles, by recycling, in the process,
of condensable light compounds: in particular, water generated by the reaction for the oxidation of propylene, unconverted acrolein, light aldehydes, such as formaldehyde and acetaldehyde, acids, such as acetic acid, the main impurity generated in the reaction section,
of heavy compounds; furfuraldehyde, benzaldehyde, maleic acid, maleic anhydride, benzoic acid, 2-butenoic acid, phenol, and the like.

The second stage of the manufacture, corresponding to stage b) of the process according to the invention, consists in recovering the acrylic acid present in the gaseous effluent stream resulting from the oxidation reaction.

This stage can be carried out by countercurrentwise absorption. For this, the gas resulting from the reactor is introduced at the bottom of an absorption column, where it encounters, countercurrentwise, a solvent introduced at the column top. The light compounds, under the temperature and pressure conditions normally employed (respectively more than 50° C. and less than 2×10⁵ Pa), are removed at the top of this absorption column. The solvent employed, in this column is water. The water could be replaced by a hydrophobic solvent having a high boiling point, as is described, for example, in the BASF patents FR 2 146 386 or U.S. Pat. No. 5,426,221, and in the patent FR 96/14397.

The operating conditions of this absorption stage are as follows:

The gaseous reaction mixture is introduced at the column bottom at a temperature of between 130° C. and 250° C. The water is introduced at the column top at a temperature of between 10° C. and 60° C. The respective amounts of water and gaseous reaction mixture are such that the water/acrylic acid ratio by weight is between 1/1 and 1/4. The operation is carried out at atmospheric pressure.

An aqueous mixture of acrylic acid in water (ratio by weight from 1/1 to 4/1) freed from most of the unconverted acrolein and light compounds, in particular noncondensable light compounds, including CO, is thus obtained, this mixture generally being referred to as “crude acrylic acid”.

This crude acrylic acid is then subjected to a combination of stages which can differ in their sequence according to the process; dehydration, which removes the water and the formaldehyde (dehydrated acrylic acid), removal of the light products (in particular the acetic acid), removal of the heavy products, optionally removal of certain impurities by chemical treatment.

The gas stream exiting from the preceding stage of extraction of the acrylic acid by countercurrentwise absorption, which consists mainly of unconverted propylene, unconverted oxygen, propane, CO and CO₂, and other minor inert gases or light impurities, is brought into contact with a solid catalyst capable of oxidizing carbon monoxide to carbon dioxide at a chosen temperature, in a fluidized bed, resulting in a stream depleted in carbon monoxide which can be recycled to the reaction stage, optionally after having bled it of a portion of the carbon dioxide formed.

In an alternative form of the process of the invention, all or part of the gas stream exiting from the unit for the conversion of carbon monoxide to carbon dioxide is sent to a selective permeation unit in order to separate a first stream predominantly comprising the inert compounds, such as CO, CO₂, nitrogen and/or argon, and a second stream predominantly comprising propylene and propane. The permeation unit employs one or more semipermeable membranes having the property of separating the inert compounds from the hydrocarbons. This separation generally takes place at a pressure of the order of 10 bar and at a temperature of approximately 50° C. Use may be made of membranes based on hollow fibers composed of a polymer chosen from: polyimides, polymers of cellulose derivatives type, polysulfones, polyamides, polyesters, polyethers, polyetherketones, polyetherimides, polyethylenes, polyacetylenes, polyethersulfones, polysiloxanes, polyvinylidene fluorides, polybenzimidazoles, polybenzoxazoles, polyacrylonitriles, polyazoaromatics and the copolymers of these polymers.

Said second stream enriched in propylene and propane is advantageously recycled to the reaction stage without accumulation of CO₂ and other inert gases, such as argon, in the recycling loop.

In a second alternative form of the process of the invention, all or part of the gas stream entering the unit for the conversion of carbon monoxide to carbon dioxide is sent beforehand to a selective permeation unit, such as that described above, to separate at least a portion of the CO₂, the gas stream entering the CO converter then being depleted in CO₂ and unconverted oxygen.

Other characteristics and advantages of the invention will become apparent in the experimental part which will follow.

### EXPERIMENTAL PART

### Example 1

Oxidation tests were carried out on pure compounds using a catalyst from Johnson Matthey (2% Pt on CeO₂) in a reactor comprising a bath of molten salt, with an internal diameter of 25.4 mm and with a catalyst height of 30 cm (i.e., 164 g).

The test consisted in monitoring the conversion of the pure compound (conversion test where each constituent is tested individually) in a mixture of nitrogen and oxygen (3 mol %). For some tests, a portion of the nitrogen was replaced by water (20 mol %). The following concentrations (which represent the order of magnitude of the concentrations expected for each of the reactants in a real stream) were tested under SV conditions of 25 000 h⁻¹, the conversion of the compound being determined as a function of the temperature:
CO: 2.8 mol %
Propylene: 0.75 mol %
Acrolein: 0.75 mol %
Propane: 50 mol %
optionally water

These conditions correspond to conditions for the oxidation of propylene in the presence of propane as thermal ballast.

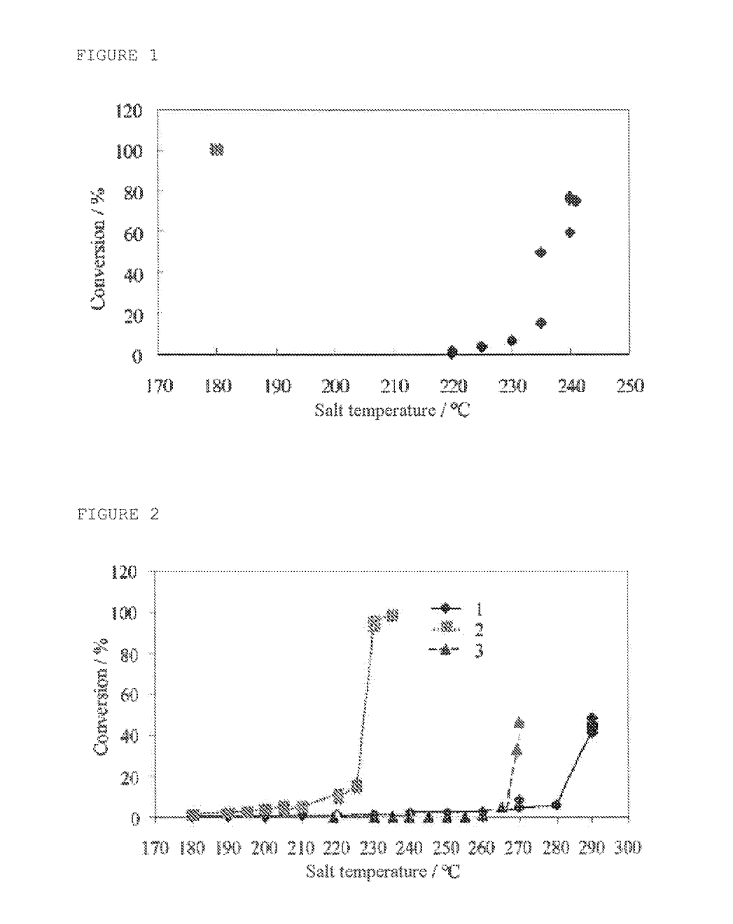

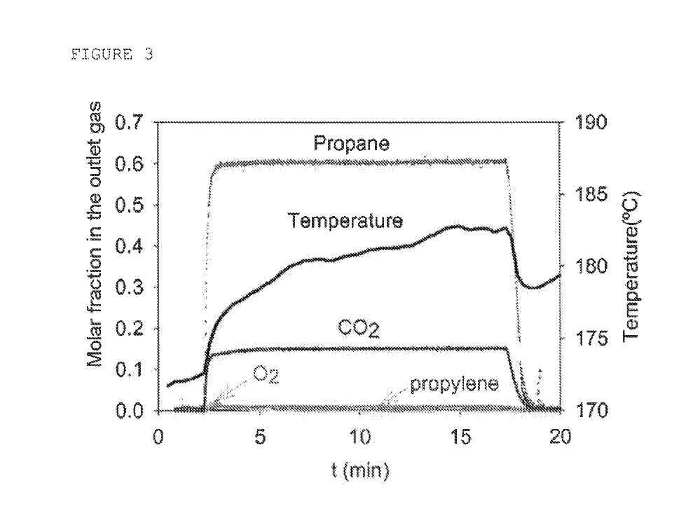

The change in the oxidation of the pure compounds as a function of the temperature, respectively in the presence of water and in the absence of water, is reproduced in FIGS. 1 and 2. In FIG. 1, the conversion of the CO is 100% for a temperature of 180° C. and the oxidation of the propylene begins significantly at a temperature of 235° C. In FIG. 2, curve 1 corresponds to acrolein, curve 2 to CO and curve 3 to propylene.

The “ignition” temperatures corresponding to the start of the oxidation reaction for the pure compounds are collated in table 2 below. In this table, the temperatures shown correspond to the temperatures applied to the reactor and not those of the catalyst.

TABLE 2

| Compounds | CO | Propylene | Acrolein | Propane |
|---|---|---|---|---|
| Ignition temperature | 225° C. | 265° C. | 285° C. | |
| without water | | | | |
| Ignition temperature | <180° C. | 235° C. | | >300° C. |
| with water | | | | |

With the pure compounds, the ignition temperature of the reaction is significantly different (difference >30° C.), showing an advantage for sufficient evacuation of the energy to keep the reaction temperature at temperatures where the selectivity is good.

### Example 2

### Comparative

Example 1 is reproduced with the fixed bed of catalyst and a stream comprising the mixture of the compounds CO, CO₂, propane, propylene, acrolein, water and oxygen with the following concentrations, in which it is desired to selectively oxidize the CO to CO₂:
CO: 2.8 mol %
Propylene: 0.75 mol %
Acrolein: 0.75 mol %
Water: 20 mol %
Oxygen: 3 mol %
Propane: 50 mol %

The tests carried out show that, in the presence of the mixture of compounds, complete combustion of the reactants is observed as long as the oxygen is available, in the following order of reactivity:
CO>propylene>acrolein>propane

The temperature is not controlled: a hot spot can be measured where the temperature reached in the catalytic bed is greater by 150° C. at least than that of the oven. Consequently, this difference being much greater than that measured between the ignition temperatures of the pure substances, all the oxidation reactions are stressed at the same time, increasing even more the overall exothermicity. The oxidation reactions are found to be slowed down only when the oxygen has been consumed.

The test was reproduced with dilution of the catalyst by 90% by weight with inert materials (alumina/silica beads originating from Saint-Gobain). The diluting of the catalyst has the object of reducing the catalytic activity of the reactor in order to exert better control over its operating temperature. Thus, the reactor was filled with 10% by weight of the initial catalyst and 90% by weight of inert materials for an equivalent volume of catalyst+inert materials. The aim is also to distribute the heat, from the oxidation reaction over a greater reaction volume. The heat from the reaction is removed by heat transfer by the wall; the inert solid provides a greater number of points of contact between the catalyst and the wall and should thus make possible better control of the temperature in the catalytic bed. Despite high dilution of the catalyst (which amounts to a strong increase in the space velocity), the difference in temperature between the hot spot, of the catalytic bed and the temperature of the oven (or the ignition temperature of the reaction of the oxidation of CO) remains greater than the difference in ignition temperature of CO and propylene.

Under these conditions, the oxidation reaction is not always controlled and the diluting of the catalyst with inert materials (90% by weight) does not make it possible to solve the problem. Here again, the catalyst is inadequate for the reactor technology used.

### Example 3

### Preparation of Catalysts for the Selective Oxidation of Carbon Monoxide

### Preparation of a Catalyst A

200 g of porous silica spheres with a diameter of 80 microns are impregnated, by nascent humidity impregnation, with a solution comprising 10.2 g of citric acid, 20.2 g of tetraammineplatinum(II) hydrogencarbonate (comprising 50.6% of platinum), 8 g of iron(III) nitrate nonahydrate and 103.5 g of demineralized water. Gentle heating with stirring is used to evaporate the excess water, the solid being kept rotated in a rotating oven in order to prevent, agglomeration. Finally, the powder is dried at 105° C. and then calcined under air at 500° C. for 2 hours.

### Preparation of a Catalyst B

A catalyst is prepared by impregnation of Puralox® SCCA 5-150 alumina from Sasol according to the following protocol:

300 g of alumina are introduced into a 3 l jacketed reactor heated to 100° C. and flushing with air is carried out in order to fluidize the alumina. A solution of 15.3 g of citric acid, 30.3 g of tetraammineplatinum(II) hydrogencarbonate (comprising 50.6% of platinum), 12 g of iron nitrate nonahydrate and 155 g of demineralized water is then continuously injected using a pump. The ratio targeted (weight of metal/weight of final catalyst) being 0.5% Pt-0.5% Fe % by weight, the duration of addition of the solution is 2 h. The catalyst is subsequently left at 105° C. in an oven for 16 h and then calcined at 500° C. for 2 hours.

This alumina has, at the start, grains having a median diameter of approximately 85 μm and exhibits the surface and porosity characteristics indicated, below:

BET surface (m²/g) 148
Kg total pore volume (cm³/g) 0.87
D50: apparent mean diameter of 50% of the population of the particles: 85 μm
Porosity peak (nm): 9

### Example 4

Example 2 is reproduced but using a fluidized bed.

The catalysts A and B are employed in a fluidized bed supplied with a stream having the composition described in table 3 below and preheated to 100° C. This stream provides for the fluidization of the catalyst. The total pressure in the reactor is 2.2 bar, with a linear velocity of the gases of 10 cm/s.

TABLE 3

| Molar composition | % |
|---|---|
| Acetaldehyde | 0.11% |
| Acrolein | 0.24% |
| H₂O | 2.43% |
| O₂ | 10.26% |
| Argon | 1.99% |
| CO | 3.95% |
| CO₂ | 23.90% |
| Propane | 53.87% |
| Propylene | 1.67% |
| Acetic acid | 0.00% |
| Acrylic acid | 0.01% |
| Nitrogen | 1.56% |
| Pressure (bar) | 2.2 |
| Temperature (° C.) | 40 |

The products are collected at the reactor outlet and are analyzed by chromatography after having condensed the water.

The results obtained appear in table 4 below:

TABLE 4

| | Conversion | Conversion | Conversion | Conversion |
|---|---|---|---|---|
| | of the | of the | of the | of the |
| Catalyst | CO (%) | propylene (%) | acrolein (%) | propane (%) |
| A | 90 | 5 | 3 | 1 |
| B | 100 | 4 | 1 | na |

### Example 5

The catalysts C, D and E defined, below are obtained in the form of granules and were milled to a particle size of less than 315 microns. 150 g of this powder are sieved in order to select the fraction between 80 and 160 microns.

Catalyst C: NO-520 catalyst from N.E. Chemcat, Pt/alumina, in the form of 3 mm beads, with a density of 0.74 kg/l.

Catalyst D: DASH 220 catalyst from N.E. Chemcat, 0.5% Pt/alumina, in the form of 3.2 mm granules, with a specific surface of 100 m²/g.

Catalyst E: ND103 catalyst from N.E. Chemcat, 0.5% Pd/alumina, in the form of beads with a diameter of 3 mm, with 250 m²/g.

150 g of solid catalyst were placed in a fluidized bed. The fluidized bed consists of a stainless steel tube with a diameter of 41 mm and a total height of 790 mm. The fluidized bed is immersed in a fluidized sand, bath heated by electrical elements installed inside the bath. Three thermocouples recorded the temperature gradient along the tube. A stream, with the molar composition described in table 5 below was supplied at a flow rate of 1760 ml/min (standard conditions), i.e. a linear velocity of the gases of approximately 2.2 cm/s, below a porous metal plate which distributes the gas across the diameter of the reactor.

The total pressure in the fluidized bed is 1 bar and the temperature is maintained at approximately 100° C.

TABLE 5

| Molar composition of the gas | |
|---|---|
| entering the fluid bed | % |
| Acetaldehyde | 0.12% |
| Acrolein | 0.31% |
| H₂O | 2.63% |
| O₂ | 4.39% |
| Argon | 1.95% |
| CO | 5.31% |
| CO₂ | 13.34% |
| Propane | 68.73% |
| Propylene | 1.93% |
| Acetic acid | 0.00% |
| Acrylic acid | 0.01% |
| Nitrogen | 1.24% |
| Pressure (bar) | 1 |
| Temperature (° C.) | 50 |

The products are collected at the fluidized bed outlet and, after having condensed the water, are analyzed by liquid chromatography.

The results obtained appear in table 6 below:

TABLE 6

| | Conversion | Conversion | Conversion | Conversion |
|---|---|---|---|---|
| | of the | of the | of the | of the |
| Catalyst | CO (%) | propylene (%) | acrolein (%) | propane (%) |
| C | 82 | 3 | 1 | 0 |
| D | 90 | 4 | 1 | 0 |
| E | 65 | 3 | 1 | 0 |

### Example 6

### Preparation of Catalysts

Three series of tests were carried out starting from the following catalysts:
0.5Pt catalyst (corresponding to 0.5% Pt and 0.5% Fe/on alumina Al₂O₃), bulk density 0.78 g/ml, mean particle size 114 μm.
1.5Pt catalyst (corresponding to 1.5% Pt and 1.5% Fe/on alumina Al₂O₃), bulk density 0.79 g/ml.
2Pd catalyst (corresponding to 2% Pd/on zeolite D/UR from Engelhard/BASF), bulk density 0.55 g/ml, mean particle size 144 μm.

The first two catalysts were prepared according to the protocols below and the third catalyst is a catalyst sold by BASF. The latter was used as is without specific modification apart from a predrying.

The catalysts for the selective oxidation of CO were prepared by impregnation of the precursors in solution (Pt and Fe salts) on the Sasol alumina Puralox SCCA 5-150.

1) In a first step, the catalysts for micro-fluidized bed tests were prepared by nascent humidity impregnation.

The platinum and iron salts and also citric acid were weighed out and dissolved in a beaker with water. The 0.5% Pt/0.5% Fe/alumina catalyst was prepared by mixing 0.049 g of citric acid, 0.0987 g of tetraammineplatinum(II) hydrogencarbonate (comprising 50.6% of Pt), 0.36 g of ferric nitrate hydrate, 8.10 g of water and 9.9 g of alumina. The total volume of the solution was calculated in order to be equal to the total pore volume of the support (0.87 ml/g). The solution was subsequently gradually added to the support while stirring vigorously. It was completely incorporated by the support and no liquid was detectable at the surface or between the particles. The resulting paste was dried at 110-120° C. for 24 h and then calcined in the air at 485-520° C. for 2 hours.

2) For the tests in a larger reactor, another technique was employed where the impregnation took place in a fluidized bed, the method described above not being very applicable to 200 g of catalyst, in particular for reasons of homogeneity. The following method was used to prepare 200 g of 0.5% Pt/0.5% Fe/Al₂O₃. The same precursor salts and the same concentrations were used as for the preparations described, above.

The solution of precursor and of citric acid is supplied dropwise above the support placed in a cylindrical quartz tube heated to 105-125° C. and fluidized with air. The liquid flow rate was calculated and maintained at 0.5-1 ml/min. The injection nozzle was placed at approximately 10 cm above the bed. The air was used as fluidization gas and the flow rate was adjusted in order to maintain an ebullating bed state and also in order to prevent pneumatic transportation of the particles. The solid thus obtained was dried at 120° C. for 24 h and then calcined at 500° C.

Two levels of charging were carried out for the tests in a microreactor (10 grams of 1.5Pt/1.5% Fe/Al₂O₃ and 10 grams of 0.5Pt/0.5% Fe/Al₂O₃) and just one for the other tests in the larger reactor (200 grams of 0.5Pt/0.5% Fe/Al₂O₃).

### Example 7

### Tests in the Fluidized Microreactor Starting from the Catalysts of Example 6

Use was made of a microreactor consisting of a quartz tube with an internal diameter of 7 mm in which 1 g of catalyst was arranged on a sintered glass (20 μm) placed in the middle of the tube acting as distributor. The entire assembly is computer controlled and it is possible to record all the relevant experimental parameters (temperatures, gas flow rates).

The tests were carried out under 40 ml/min for 15 min. This corresponds to a space velocity SV of approximately 3000 h⁻¹.

The composition of the inlet gas supplied to the microreactor is as follows:

| Gas | Ar | O₂ | C₃H₆ | CO | CO₂ | C₃H₈ |
|---|---|---|---|---|---|---|
| % vol. | 5.53% | 7.61% | 1.69% | 5.19% | 10.6% | 66.79% |

Between two tests, 40 ml/min of argon were sent in order to purge the reactor for 10 min. Argon is also the inert material of choice during the heating periods.

In order to have an idea of the exothermicity of the oxidation reaction, monitoring of the temperature was carried out on the temperature of the oven and not on that of the catalyst bed. For each experiment carried out, the operating conditions were kept constant during the 15 min of the test.

The gases at the outlet of the reactor were analyzed by an inline mass spectrometer.

FIG. 3 is presented as an example and gives the composition of the outlet gas after reaction and also the change in the temperature of the catalytic bed. All the experiments carried out give the same type of profile with stationary levels. It is from these data that a mean conversion was calculated.

The tests carried out in the microreactor are summarized in table 7 below.

TABLE 7

| | | | | Temperature | | | |
|---|---|---|---|---|---|---|---|
| | | Temperature of | Maximum | of the oven | | | |
| | | the bed at the start | ΔT of the | (° C.) | X CO | X propylene | X propane |
| Test | Catalyst | of the test (° C.) | bed (° C.) | (constant) | (%) | (%) | (%) |
| 1 | 0.5Pr | 112 | 8 | 100 | 4 | 0 | 0 |
| 2 | | 120 | 11 | 110 | 47 | 0 | 0 |
| 3 | | 134 | 11 | 120 | 62 | 0 | 0 |
| 4 | | 148 | 8 | 130 | 78 | 0 | 0 |
| 5 | | 160 | 10 | 140 | 90 | 0 | 0 |
| 6 | | 172 | 10 | 150 | 100 | 0 | 0 |
| 7 | | 183 | 12 | 160 | 100 | 0 | 0 |
| 8 | | 195 | 14 | 170 | 100 | 0 | 0 |
| 9 | | 206 | 13 | 180 | 100 | 1 | 5 |
| 10 | | 217 | 36 | 190 | 100 | 34 | 12 |
| 11 | | 228 | 32 | 200 | 100 | 49 | 27 |
| 12 | 1.5Pt | 117 | 8 | 100 | 54 | 0 | 0 |
| 13 | | 129 | 7 | 110 | 82 | 0 | 0 |
| 14 | | 140 | 8 | 120 | 100 | 0 | 0 |
| 15 | | 152 | 18 | 130 | 100 | 13 | 8 |
| 16 | | 163 | 33 | 140 | 100 | 41 | 10 |
| 17 | | 173 | 36 | 150 | 100 | 43 | 10 |
| 18 | | 185 | 38 | 160 | 100 | 43 | 10 |
| 19 | | 196 | 38 | 170 | 100 | 45 | 10 |
| 20 | | 207 | 35 | 180 | 100 | 45 | 10 |
| 21 | | 219 | 32 | 190 | 100 | 45 | 10 |
| 22 | | 229 | 29 | 200 | 100 | 45 | 10 |
| 23 | 2Pd | 118 | n.a. | 100 | 92 | 0 | 0 |
| 24 | | 131 | n.a. | 110 | 100 | 0 | 0 |
| 25 | | 142 | n.a. | 120 | 100 | 0 | 0 |
| 26 | | 155 | n.a. | 130 | 100 | 2 | 0 |
| 27 | | 161 | n.a. | 140 | 100 | 5 | 0 |
| 28 | | 170 | n.a. | 150 | 100 | 11 | 0 |
| 29 | | 187 | n.a. | 160 | 100 | 12 | 0 |
| 30 | | 107 | n.a. | 170 | 100 | 13 | 0 |
| 31 | | 208 | n.a. | 180 | 100 | 11 | 0 |
| 32 | | 219 | n.a. | 190 | 100 | 23 | 0 |
| 33 | | 235 | n.a. | 200 | 100 | 20 | 0 |

n.a. for not available

The lines in bold characters are the experiments for which the oxidation of CO was complete and selective.

These tests have shown that the selective oxidation of CO in the fluidized bed is highly efficient, with conversions of 100% and the absence of oxidation of propylene and propane, at well controlled temperatures, since the increase in temperature in the bed was minimal.

It is therefore entirely possible to envisage the selective oxidation of CO in a fluidized bed on a larger scale provided that mild oxidation conditions and good heat transfer are provided. Starting from mathematical principles using kinetic constants, it is then entirely possible to determine the optimum operating conditions to be employed on the industrial scale.

### Example 8

### Tests in a Fluidized Bed Reactor

A fluidized bed of greater size makes it possible to carry out tests on amounts of catalyst which have reached 300 g. The metal reactor is divided into two sections; a reactive section (diameter 3.5 cm, height 60 cm) and a withdrawal region (diameter 4.5 cm, height 40 cm). The gases are supplied and regulated via flow regulators. These make it possible to achieve a maximum flow rate by volume in this plant of 4 l/min. Control of the temperature is provided by an external sand bath which makes it possible to provide good homogeneity of the temperature in the reactor. Four measurement points for the temperature were installed inside the reactor (two in the catalytic bed and two in the withdrawal section). A valve was also installed on the outlet line in order to be able to raise the reactor up to the desired pressure. A pump of HPLC type introduces an unchanging flow of an aqueous acrolein solution directly into the catalytic bed.

An inline mass spectrometer makes it possible to quantify the products. A line heated to 110° C. connects the mass spectrometer to the assembly and thus prevents any condensation of water. Filters installed on the outlet line prevent the particles from exiting from the reactor and prevent the lines from becoming blocked.

Four different gas compositions were tested and are summarized in the following table 8. The tests denoted T1 were carried out under 2.2 bar absolute, while the tests denoted T2 were carried out at atmospheric pressure. Each Test, T1 or T2, was carried out “dry” or “wet” (in the presence of water vapor).

TABLE 8

| In % | Ar | O₂ | C₃H₆ | CO | CO₂ | C₃H₈ | C₃H₄O | H₂O |
|---|---|---|---|---|---|---|---|---|
| T1 dry | 3.8 | 11.3 | 1.7 | 4.4 | 23.9 | 54.9 | 0 | 0 |
| T1 wet | 3.4 | 10.1 | 1.5 | 4.0 | 21.4 | 49.2 | 2.7 | 7.7 |
| T2 dry | 3.2 | 4.5 | 1.8 | 5.5 | 14.0 | 71.0 | 0 | 0 |
| T2 wet | 2.9 | 4.0 | 1.6 | 4.9 | 12.6 | 63.6 | 3.0 | 7.3 |

The tests were carried out on 150 g of 0.5Pt catalyst prepared according to example 6. The flow rate by volume of the gases was maintained at approximately 600 ml/min for 15 to 40 minutes (depending on the test and on the time for stabilization of the outlet concentrations) for the tests and for the purges with argon in order to provide good fluidization of the particles. This represents an hourly space velocity of approximately 180 h⁻¹. It was not possible to operate with higher gas flow rates, in order to achieve much shorter contact times and a greater hourly space velocity, without bringing about entrainment of the catalyst as a result of the small diameter of the reactor. Nevertheless, these results are representative of the tests which a person skilled in the art is capable of extrapolating for a unit of greater size, with greater gas linear velocities and greater hourly space velocities.

A mean conversion was calculated, over the whole of the 15-40 minutes of the test.

The results for test 1, carried out under a pressure of 2.2 bar, and test 2, carried out under a pressure of 1 bar, are summarized in tables 9 and 10 respectively.

TABLE 9
Test 1 (2.2 bar)

| | Temperature of | Maximum | Duration | | Conversion | Conversion |
|---|---|---|---|---|---|---|
| | the bed at the | ΔT of | of the | Conversion | of the | of the |
| Test 1 | start of the test | the bed | experiment | of the | propylene | propane |
| 2.2 bar | (° C.) | (° C.) | (min) | CO (%) | (%) | (%) |
| Dry | 100 | 3 | 35 | 27 | 10 | 1 |
| Dry | 110 | 43 | 30 | 100 | 17 | 1 |
| Wet | 100 | <1 | 25 | 16 | 1 | 2 |
| Wet | 110 | 48 | 40 | 100 | 26 | 5 |

TABLE 10
Test 2 (1 bar)

| | Temperature of | Maximum | Duration | | Conversion | Conversion |
|---|---|---|---|---|---|---|
| | the bed at the | ΔT of | of the | Conversion | of the | of the |
| Test 2 | start of the test | the bed | experiment | of the | propylene | propane |
| 1 bar | (° C.) | (° C.) | (min) | CO (%) | (%) | (%) |
| Dry | 100 | 2 | 15 | 39 | 0 | 0 |
| Dry | 120 | 4 | 20 | 93 | 0 | 0 |
| Dry | 140 | 4 | 20 | 100 | 0 | 0 |
| Wet | 110 | 1 | 15 | 27 | 1 | 1 |
| Wet | 120 | 1 | 15 | 12 | 1 | 1 |

The duration of the reaction was adjusted so as to achieve a stationary state for the outlet concentrations.

No conversion of propylene or of propane was observed under 1 bar, even at temperatures above 140° C. Furthermore, the increase in temperature in the bed was very limited, indicating very good heat transfer in the fluidized bed.

1. A process for the selective oxidation of carbon monoxide present in a gas mixture comprising at least one hydrocarbon or one hydrocarbon derivative and carbon monoxide, said process comprising bringing said gas mixture into contact with a fluidized bed of a solid oxidizing catalyst whereby said carbon monoxide is oxidized to carbon dioxide at a chosen temperature.

2. The process as claimed in claim 1, characterized in that the hydrocarbon is a saturated or mono- or diunsaturated linear or branched hydrocarbon comprising from 2 to 6 carbon atoms, or an aromatic hydrocarbon comprising from 6 to 12 carbon atoms.

3. The process as claimed in claim 1, characterized in that the hydrocarbon derivative is selected from the group consisting of anhydrides, aldehydes, unsaturated carboxylic acids, unsaturated nitriles, mixtures thereof, ethylene oxide, propylene oxide and 1,2-dichloroethane.

4. The process as claimed in claim 1, characterized in that the catalyst is in the form of solid particles with a particle size ranging from 20 to 1000 microns.

5. The process as claimed in claim 1, characterized in that the catalyst is selected from the group consisting of catalysts based on noble metals supported on an inorganic support.

6. The process as claimed in claim 1, characterized in that the temperature of the fluidized bed is lower than the temperature corresponding to the start of a reaction for the oxidation of the hydrocarbon and/or hydrocarbon derivative.

7. The process as claimed in claim 1, characterized in that the space velocity SV, expressed as hourly flow rate by volume of gas mixture with respect to the volume of catalyst, is between 1000 h⁻¹ and 30 000 h⁻¹.

8. A process for the production of a hydrocarbon derivative comprising oxidizing carbon monoxide present in a gas mixture comprising at least one hydrocarbon or one hydrocarbon derivative and carbon dioxide in a fluidized bed comprising a solid catalyst capable of oxidizing carbon monoxide to carbon dioxide at a chosen temperature.

9. (canceled)

10. The process as claimed in claim 23, characterized in that all or part of the gas mixture entering or the gas mixture depleted in carbon monoxide exiting from stage c) is subjected to a separation in a selective permeation unit, whereby at least a portion of the carbon dioxide present in said gas mixture entering or the gas mixture depleted in carbon monoxide exiting from stage c) is removed.

11. The process as claimed in claim 23, characterized in that stage a) is carried out in the presence of a thermal ballast which is inert under the conditions of the process.

12. The process as claimed in claim 23, characterized in that the hydrocarbon is propylene and the hydrocarbon derivative is acrylic acid.

13. The process as claimed in claim 11, characterized in that propane is said thermal ballast.

14. The process as claimed in claim 2, characterized in that said aromatic hydrocarbon is substituted.

15. The process as claimed in claim 3, characterized in that said anhydride is selected from the group consisting of phthalic anhydride and maleic anhydride.

16. The process as claimed in claim 3, characterized in that said aldehyde is selected from the group consisting of acrolein and methacrolein.

17. The process as claimed in claim 3, characterized in that said unsaturated carboxylic acid is selected from the group consisting of acrylic acid and methacrylic acid.

18. The process as claimed in claim 3, characterized in that said unsaturated nitrile is selected from the group consisting of acrylonitrile, methacrylonitrile and atroponitrile.

19. The process as claimed in claim 4, characterized in that the catalyst is in the form of solid particles with a particle size ranging from 40 to 500 microns.

20. The process as claimed in claim 5, characterized in that the noble metal is selected from the group consisting of platinum, palladium, rhodium and ruthenium.

21. The process as claimed in claim 5, characterized in that the inorganic support is selected from the group consisting of silica, titanium oxide, zirconium oxide, alumina, sodium silicate and silicalite.

22. The process as claimed in claim 1 characterized in that the catalyst is selected from the group consisting of copper, manganese, cobalt, nickel and iron, optionally in the presence of at least one noble metal in the form of mixed oxides or of alloys, optionally supported on an inorganic support.

23. A process for the production of a hydrocarbon derivative comprising:
a) bringing at least one hydrocarbon and oxygen or an oxygen-comprising gas into contact with a catalyst to form a gas mixture comprising at least one hydrocarbon derivative, unconverted hydrocarbon, oxygen and carbon monoxide,
b) separating or extracting the hydrocarbon derivative from the gas mixture from stage a),
c) converting the carbon monoxide present in the gas mixture to carbon dioxide in a fluidized bed comprising a solid catalyst capable of oxidizing carbon monoxide to carbon dioxide at a chosen temperature, to produce a gas stream, depleted in carbon monoxide,
d) recycling said gas stream depleted in carbon monoxide to reaction stage a).
